(19) United States
(12) Patent Application Publication
(10) Pub. No.: US 2021/0316126 A1
(43) Pub. Date: Oct. 14, 2021

(54) Seal

(71) Applicant: Spectrum Medical Ltd., Gloucester, Gloucestershire (GB)

(72) Inventor: Jeremy Tamari, Gloucester, Gloucestershire (GB)

(21) Appl. No.: 17/284,554

(22) PCT Filed: Oct. 2, 2019
(86) PCT No.: PCT/GB2019/052770

§ 371 Date: Apr. 12, 2021

(30) Foreign Application Priority Data
Oct. 12, 2018 (GB) 1816677.7

(51) Int. Cl. A61M 39/02

(52) U.S. Cl. CPC A61M 39/0247; A61M 2039/0258; A61M 2039/027; A61M 2039/0273; A61M 2039/0282; A61M 2039/0261; A61M 2039/0291

(57) ABSTRACT

The invention relates to a vascular access device (10) for implantation onto a vessel wall, the access device comprising an tubular structure (12) to provide an access passage into the vessel via a hole in a vessel wall, a connector arrangement (20; 21) for attaching the tubular structure at said hole in the vessel wall, and a membrane structure (16A; 16B) sufficiently flexible to be collapsed for insertion through said hole and to be expanded to lie at least partially against an inner vessel surface thereby to surround the hole. The membrane structure comprises a membrane aperture (18) to provide a passage from inside the vessel into the tubular structure. The vascular access device further comprises a closing mechanism capable of closing the membrane aperture while the membrane structure is expanded against said inner vessel surface. There is also provided a method of implanting a vascular access device.

### FIELD OF THE INVENTION

The present invention relates to a vascular access device, vascular access method and corresponding system. More specifically, the present invention relates to a vascular access device comprising a port that is implantable onto a vessel wall, and a method of using such a device for vascular access.

### BACKGROUND

During extracorporeal life support, such as extracorporeal membrane oxygenation, a typical surgical protocol includes draining and re-introducing fluids such as blood from and into a patient. One of the limitations to be considered by a clinician is the suitability of the access site, e.g. the diameter of the vessel to accommodate channels for both drainage and infusion or location of the access site on the patient and relative to the target organ. Clinicians often face a need to limit the repertoire of techniques based on suitability of an insertion technique, vessel site, vessel size, and obstruction of distal perfusion, e.g. a need to avoid a situation in which a medical instrument inside a blood vessel unduly restricts blood flow that would otherwise supply areas downstream of the medical instrument. Furthermore, a clinician may have to consider practicalities such as how far from the target organ the insertion can be made to be effective. As one example, for infusion towards the heart from a vessel downstream of the heart, it may have to be considered that the heart may pump against the infusion direction.

So-called dual-lumen cannulas alleviate the problem of accommodating two channels to some extent by way of a single cannula that comprises one lumen suitable for drainage and another lumen suitable for infusion. A limitation of such devices is that their cumulative cross-sections are smaller than that of the vessel into which they are inserted.

The present invention seeks to further increase the repertoire of vascular access devices available to clinicians.

### SUMMARY OF THE INVENTION

In accordance with a first aspect of the invention, there is provided a vascular access device as defined in claim 1.

The vascular access device is of the type for implantation onto a vessel wall and comprises a tubular structure, a connector arrangement and a membrane structure.

The tubular structure is to provide an access passage into the vessel via a hole in a vessel wall. The connector arrangement is for attaching the tubular structure at said hole in the vessel wall. The membrane structure is sufficiently flexible to be collapsed for insertion through said hole and to be expanded to lie at least partially against an inner surface of a vessel thereby to surround the hole. The membrane structure comprises a membrane aperture to provide a passage from inside the vessel into the tubular structure. The vascular access device further comprises a closing mechanism capable of closing the membrane aperture while the membrane structure is expanded against said inner surface.

It will be understood that a blood vessel wall may be accessible percutaneously, by inserting a device such as a cannula or “needle” through the skin until it reaches the blood vessel wall, or more directly after a surgical cut-down to the vessel. The vascular access device of the present invention is intended for implantation onto a vessel wall. To this end, a wall of the vessel is cut or punctured to create a hole that can be widened to a diameter, depending on vessel diameter, of typically a few millimetres or “French” (1 millimetre=3 French). To provide context, the diameter of an axillary artery is typically in the region of 5 to 8 mm (left axillary arteries being slightly smaller than right axillary arteries), and the diameter of a femoral artery is in the region of 6 to 10 mm (left femoral arteries being slightly smaller than right femoral arteries) and so a hole of a few millimetres diameter or more constitutes a significant puncture.

The membrane structure is insertable through the vessel via such a hole, made via an incision or puncture, into the inside of the blood vessel and expandable to provide a membrane covering the hole from the inside. The membrane may be a structure such as a patch of suitable material such a silicone, PTFE (polytetrafluorethylene) or other tissue-like materials such as silk. The membrane structure may be provided with active agents or surface treatment to provide anti-bacterial properties and/or anti-coagulant properties. The width of the patch corresponds to the vessel into which it is intended to be inserted and may, in expanded condition, extend no more than a few centimetres. For instance, the patch may have a longest diameter of no more than about 6, 7, 8 or 9 cm.

The membrane structure comprises a membrane aperture that can be readily aligned with the hole in the vessel wall such that the membrane aperture provides a passage between the vessel inside and outside. The membrane surrounds the membrane aperture and lies against the inner surface of the vessel wall to practically seal the hole in the vessel wall such that flow between the membrane and the vessel wall towards the vessel hole is practically inhibited while flow is possible via the hole through the membrane aperture when open.

The vascular access device may comprise anchoring structures such as feet or a ring structure that are also insertable into the vessel through the hole and expandable and/or articulated to provide structural support to the membrane.

The connector arrangement may comprise a rigid or pliable structure such as an annular structure connected or connectable to either or both the membrane structure and the tubular structure. The connector arrangement allows the tubular structure to be attached near the hole and thereby facilitates alignment and/or location with the membrane aperture, to provide an access passage via the tubular structure and the membrane aperture into the blood vessel. The connector arrangement assists with improving the fluid-tightness of the connection between the membrane structure and the tubular structure. The connector arrangement may be provided by correspondingly shaped coupling structures, such as structures providing a friction fit or bayonet-style engagement. The connector arrangement may be provided by adhesive locations and/or by magnetic coupling. It may comprise a friction fit coupling.

The access passage can be utilised in the manner of a cannula, i.e. providing access to the inside of a blood vessel, for instance as fluid passage or as conduit for a medical instrument such as a catheter. It is a property of the access passage that the access passage remains, apart from an end portion that may protrude through the membrane aperture, practically entirely outside the vessel wall. Thereby, the vascular access device avoids a problem with cannulas or other devices that are deployed inside a vessel, which is that a device inside a blood vessel obstructs flow in the access vessel. In contrast, the access device of the present invention requires only few structures of relatively small cross-section, such as a flat membrane along the vessel wall, and for practical purposes does not obstruct flow in the access vessel. Furthermore, the present access device does not obstruct distal perfusion of the run-off vascular network of the vessel that has been accessed.

The tubular structure can be of any length, and thereby can be dimensioned to allow access to blood vessels that may be too deep for conventional procedures, even at the femoral artery site where adipose layers can be deep.

The access device comprises closing mechanism to close the membrane aperture while the membrane structure is expanded against the vessel wall. The closing mechanism allows repeated opening and closing of the membrane aperture to provide access via the passage. Likewise, the closing mechanism can be closed to temporarily close the access passage. Compared to removal of a cannula directly inserted into a vessel, the closing mechanism of the present vascular access device allows removal of a cannula from a blood vessel with a practically automatic closure of the puncture in the vessel wall.

The present invention is based in part on the appreciation that the membrane aperture can be made relatively large, much larger than a puncture for a conventionally thin catheter, if a valve arrangement is provided as a closure mechanism that allows the membrane aperture to close one or more times.

The tubular structure may comprise a region of a sectile material. By “sectile”, it is meant that the material is cuttable with hand-operated utensils usually available in a surgical environment, such as scalpels, lancets or scissors. Thereby, the access passage can be cut to length as required by a surgeon. In particular, the tubular structure can be of a length sufficient to reach from the blood vessel to the outside skin of a patient. Depending on body location and amount of adipose tissue, the distance from blood vessel to skin may vary considerably.

By way of the sectile material, the same device can be used for different passage lengths as the device can be cut to the required length. A further property of a sectile material is that it can be sewn/sutured or clipped as required, e.g. at the end of the surgical procedure.

The tubular structure may be made entirely from sectile material. In embodiments, a portion of the access passage is made from sectile material. A typical example of a sectile material is a so-called “graft”, which may be a tube of woven, non-permeable PTFE as it is known for anastomosis (sewing onto) to blood vessels.

In embodiments, the tubular structure is rigid or comprises at least a portion of rigid material. This reduces the risk of the tubular structure inadvertently bending, thereby affecting flow through it.

The closing mechanism may comprise a cap or foam.

In some embodiments, the closing mechanism comprises a valve arrangement, wherein optionally the valve arrangement comprises one or more flaps, wherein optionally one or more of the flaps are dimensioned to overlap at least another flap.

By flap, a portion of material is meant that can be changed from a configuration that at least partially inhibits flow through an aperture into a second configuration in which flow is less inhibited. For instance, the material may at least partially overhang an aperture to restrict flow or may be bent back to reduce or remove the amount of overhanging material, to thereby remove a flow restriction.

The flaps may include a structure providing a bias to close under a pressure from the vessel lumen. It can be imagined how such flaps are easily negotiated by a device such as a catheter inserted through the access passage and into the blood vessel. In the absence of an object to be inserted, the pressure of flowing blood may suffice to urge the flaps into a closed position. In embodiments, the flaps are provided with a biasing mechanism such as a shape memory material. The biasing mechanism may urge one or more of the flaps into the closed position.

In some embodiments, the closing mechanism such as the valve arrangement comprises a shape memory material, wherein optionally the shape memory material is embedded in the membrane structure.

By providing a shape memory material, for instance a shape memory alloy such as

Nitinol, in the valve arrangement, it can be avoided that the valve arrangement loses over time its pliability to effectively seal the membrane aperture. This is believed to increase considerably the time the vascular access device can be reliably used inside a human body, from a few days to several weeks. For instance, a valve arrangement may be designed to open and close repeatedly over the course of several weeks to provide access for different instruments. As another example, the valve arrangement may be open to provide a fluid passage for extracorporeal perfusion for a prolonged period of time, for instance for several weeks. At the end of the procedure, the valve arrangement should close in a practically fluid-tight manner even though the material has been exposed to the body's immune system for several weeks or more.

The shape memory material may be embedded in the membrane structure. It may be embedded in a portion of the membrane structure sufficient to ensure the proper functioning of the valve arrangement.

For instance, the shape memory material may be provided in the form of sheets, struts or as a web, urging one or more components of the valve arrangement into a flow-arresting configuration.

The shape memory material may be embedded in parts of the membrane structure that are designed to lie against the vessel wall, for instance to assist with the expansion of the membrane structure.

In some embodiments, the closing mechanism is positioned near or at an end of the tubular structure.

The closing mechanism such as a valve arrangement may be part of the tubular structure, part of the connector arrangement, and/or part of the membrane structure. If the valve arrangement is provided as part of the membrane structure, it can be designed in a manner that the absence of the connector arrangement or the tube structure does not affect the vessel-sealing function of the valve arrangement. If the valve arrangement is provided as part of the connector arrangement and/or the tube structure, a more rigid valve arrangement may be provided.

In some embodiments, one or more of the flaps comprise an mutually engaging profiles at flap-to-flap edges, wherein preferably the mutually engaging profiles comprises corresponding tapers, corresponding tongue and groove profiles, corresponding chicane profiles and/or corresponding chevron profiles.

The mutually engaging profiles improve the fluid-tightness of the seal at the flap edges and help to ensure that adjacent flaps are locking in position to provide a seal. Likewise, for single flap, mutually engaging profiles may be provided along the flap edge and along an edge of the membrane aperture.

In some embodiments, at least part of the closing mechanism is provided by a portion of the membrane structure.

This may facilitate manufacture and/or integration of the closing mechanism such as a valve arrangement with the vascular access device. In some embodiments, the membrane aperture is provided by the location of the flaps cut into a portion of the membrane structure surrounded by integral membrane material. The flaps may overlap at least partially.

In some embodiments, the vascular access device comprises a haemostatic agent.

The haemostatic agent may be provided in the form of a surface treatment. The haemostatic agent reduces the risk of thrombosis (blood-clotting) near foreign bodies such as the components of the vascular access device. This helps to maintain the functioning of the device, e.g. pliability and tightness of the flaps over prolonged periods of time. Furthermore, the haemostatic agent helps to use the tubular structure as a flow channel for blood. For instance, a haemostatic agent may be provided on the membrane structure, and/or on the inside of the tubular structure, to assist with maintaining good blood flow properties through the tubular structures.

The haemostatic agent may lose its activity over time. The haemostatic agent may be designed or chosen to have a half-life corresponding to the intended treatment duration. For instance, the haemostatic agent may be of a composition or concentration that ensures a minimum half-life in a typical use scenario.

In some embodiments, at least part of the connector arrangement is integral with the membrane structure.

In some embodiments, at least part of the connector arrangement is integral with the tubular structure.

In some embodiments, the connector arrangement comprises a component detachably connectable to the membrane structure, optionally via correspondingly shaped coupling features.

In some embodiments, the connector arrangement provides an annular port to receive an end of the tubular structure.

The connector may be a separate component to be affixed to either the membrane structure or the tubular structure. The connector arrangement may comprise a connector component on each of the membrane structure and the tubular structure. The connector arrangement may be designed to assist with the location of the tubular structure on the membrane aperture, for instance by way of alignment features such as correspondingly shaped coupling features. One or more connector components may be integral with the membrane structure and/or the tubular structure, for instance by way of an annular rim near or at the end of the tubular structure.

In some embodiments, the tubular structure comprises an end portion whose outer circumference is smaller than the membrane aperture so as to be insertable through the membrane aperture.

The end portion may be designed to allow the tubular structure to protrude through the membrane aperture, i.e. into the blood vessel when implanted.

In some embodiments, the tubular structure comprises an end portion whose outer circumference corresponds sufficiently closely to the membrane aperture so as to be able to maintain the closing mechanism in a flow-permitting configuration.

The end portion may be designed to open the closing mechanism, for instance a valve arrangement, upon insertion into the blood vessel. For instance, it can be imagined that a valve arrangement comprised of flaps in a membrane may be pushed open by the end portion of the tubular structure, such that the flaps open, inwardly, into the blood vessel lumen. For instance, a plurality of flaps may surround the end portion.

The flaps may be biased into an aperture-closing condition, for instance by suitable orientation in relation to the blood flow direction, and/or by use of a biasing means such as a shape memory material. The end portion of the tubular structure may be wide enough to maintain the flaps in an open condition, as they are pushed inward into the blood vessel, against the bias of the flaps.

In some embodiments, the tubular structure comprises around at least part of its outer circumference a seating surface of wider circumference and set apart from an end portion of the tubular structure, wherein optionally the seating surface is of annular shape and/or provided by a plurality of feet.

The outer circumference of the end portion may be narrower than the circumference of the tubular region adjacent the end portion, to effectively provide a seating surface. This provides a defined length of the end portion and reduces the risk of an end portion being pushed in too far into the blood vessel. The seating surface may be provided by a ring structure, such as a collar, surrounding the tubular structure and/or by a stepped feature in the wall of the tubular structure. The seating surface may be provided by one or more feet structures, such as arms or flaps.

The seating surface may be collapsible to assist implantation. For instance, the seating surface may be provided in the form of a collapsible collar or a plurality of collapsible flaps radially spaced apart around the circumference of the tubular structure. The seating surface may be biased in the insertion direction of the tubular structure, such that abutment against an outer surface of the vessel wall urges the seating surface into a wider configuration, thereby resisting further introduction of the tubular structure into the vessel.

In some embodiments, at least part of the connector arrangement is provided by the seating surface.

The seating surface may be designed to cooperate with the connector arrangement or may be part of a connector cooperating with another connector part on the membrane structure.

In some embodiments, the tubular structure comprises an end portion whose inner circumference has a lumen diameter sufficient to permit flow rates of 1 litre per minute or more at typical vascular flow pressures, such as a lumen diameter of at least 2 mm, 3 mm, 4 mm, 5 mm or 6 mm, which may extend along the length of the end portion.

The end portion may be designed to allow a free flow of blood of at least 1 litre per minute (lpm), or at least 2, 3, 4 or 5 lpm or more at typical vascular driving pressures, thereby to provide a flow channel suitable for administration of flow rates in the region of 1 to 10 litres per minute (lpm). A typical vascular driving pressure may be as low as in the region of 20 mmHg and may be up to a few hundred mmHg. The lumen diameter of the tubular structure may be designed to allow the above-mentioned flow rates at such typical driving pressures.

For instance, typical perfusion rates may be in the region of a target set point in the region of 3, 4 or 5 lpm, and may be modulated in the course of an intervention to flow up to 2 lpm above and below the target flow rate. Thus, depending on the size of the flow passage, flow rates may be modulated in the region of 1-5 lpm, 2-6 lpm, or 3-7 lpm, to provide illustrative examples. To achieve such flow rates of up to 7, 8, 9 or 10 lpm, the end portion may be practically free of flow-obstructing features such as internal seating surfaces, and may have a practically homogenous diameter along practically all of its length. The lumen cross-section may be generally round or elliptical.

In some embodiments, the end portion comprises regularly repeating inner wall structures, such as spiral structures suitable to create helical flow patterns.

Such inner wall structures may facilitate a particular flow pattern, e.g. a turbulence-reducing jet, e.g. a helical flow pattern, that reduces turbulences upon introduction of fluid into the blood stream compared to the turbulences that would be observable with straight jets such as jets introduced into a blood vessel at a near-right angle.

In some embodiments, the tubular structure comprises on its outer surface a plurality of engagement structures spaced apart along the elongate extension of the tubular structure, wherein optionally the engagement structures extend circumferentially around the outer surface, and/or wherein optionally the engagement structures comprise grooves and/or ridges.

The tubular structure may be sufficiently long to extend to the outside skin of a patient. To reduce the risk of the tubular structure being pushed into the body more than intended by pushing the external end of the tube against the skin, which could cause pressure on the blood vessel at the location of the hole (the incision or puncture site), the vascular access device may be provided with an external anchor such as tape or ties. The external anchor may be provided by way of a clip or ring surrounding part or all of the tubular structure such that the tubular structure cannot be pushed further into the skin than the limit provided by the anchor. The engagement structures facilitate the positioning and repositioning of clips or rings.

The tubular structure may comprise other structures such as arms and/or flaps that can be tied or taped to the skin of a patient. Different fixation techniques may be appropriate depending on the location of the tubular structure on the body and depending on the duration of the clinical intervention.

In some embodiments, the connector arrangement comprises a pliable material suitable to adapt to the curvature of a vessel wall.

The connector arrangement may comprise pliable material, e.g. textile or a membrane, with a degree of flexibility to facilitate insertion into the body. For instance, a ring-shaped cuff of flexible material may suffice to help locating and maintaining the tubular structure at the desired location, i.e. aligned with the hole, and reduce the risk of the tubular structure detaching from the blood vessel. The material may be deformable to an extent allowing it to conform to a vessel curvature. The material may be deformable to an extend allowing it to conform to the shape of the hole in the vessel wall and the thickness in the vessel wall.

The pliable material may comprise suitable material such a silicone, PTFE (polytetrafluorethylene) or other tissue-like materials such as silk. The pliable material may be provided with active agents or surface treatment to provide anti-bacterial properties and/or anti-coagulant properties.

In some embodiments, the connector arrangement and/or the membrane structure comprises an anchor arrangement collapsible for insertion through said hole and deployable to lie at least partially against an inner and/or outer surface of a vessel, wherein preferably the anchor arrangement comprises a plurality of feet extending from the access passage.

The anchor arrangement provides a retainer structure that may comprise one or more feet or retaining elements to better hold the vascular access device on the vessel wall.

The retainer structure may be formed in the manner of splayed feet that provide an anchoring function to resist a pulling-out of the vascular access device. The retainer structure may comprise one or more clips that hold onto the rim of the hole, thereby clamping against the outside and inside of the vessel.

In some embodiments, at least part of the membrane structure and or the at least part of the connector arrangement is sufficiently biocompatible to be overgrown, thereby allowing the membrane structure to be left in place after conclusion of a surgical procedure.

In some embodiments, at least part of the membrane structure and or the at least part of the connector arrangement is dissolvable over time in situ, thereby allowing the membrane structure to be left in place after conclusion of a surgical procedure.

The design of the vascular access device may take into account the post-surgery removal of the device or parts of it. Solid components may be collapsible to facilitate removal.

Components that remain inside the vessel may not be readily removable after the vessel wall has healed. As such, the membrane structure, the valve arrangement, the connector arrangement, and/or parts thereof may be made from a material that dissolves over time and/or is sufficiently biocompatible to be overgrown (endothelialised, e.g. overgrown with vessel-lining tissue).

In some embodiments, the vascular access device is comprised in a deployment system comprising an elongate guide structure and a release mechanism capable of triggering the change of the membrane from a collapsed condition to an expanded condition.

In accordance with another aspect of the invention, there is provided a membrane component, as defined by claim 17, for use with a vascular access device according to any one of the embodiments of the first aspect.

The membrane component comprises within its membrane material one or more flaps that are flexible to open a hole in the membrane component and flexible to close the hole while the membrane structure is expanded, thereby providing a valve arrangement for the hole in the membrane component.

The hole constitutes a membrane aperture. As described in relation to embodiments of the first aspect, the membrane component, for example the flaps part of the valve arrangement, may comprise a shape memory material, wherein optionally the shape memory material is embedded in the membrane structure. One or more of the flaps may comprise mutually engaging profiles at flap-to-flap edges, wherein preferably the mutually engaging profiles are provided by corresponding tapers, corresponding tongue and groove profiles, corresponding chicane profiles and/or corresponding chevron profiles.

In accordance with another aspect of the invention, there is provided a tubular structure, as defined by claim 20, for use with a vascular access device according to any one of the embodiments of the preceding aspects. The tubular structure comprises an end portion whose inner circumference has a lumen diameter sufficient to permit flow rates of 1 litre per minute or more at typical vascular driving pressures, and further comprises around at least part of its outer circumference a seating surface of wider circumference than the end portion and set apart from the end portion. The tubular structure may comprise any combination of features described in relation to embodiments of the first or second aspect. The tubular structure may comprise and end portion whose outer circumference is smaller than the membrane aperture of the preceding aspects so as to be insertable through the membrane aperture, and/or whose outer circumference corresponds sufficiently closely in shape to the membrane aperture so as to be able to maintain the closure mechanism in a flow-permitting configuration. The seating surface is of annular shape and/or provided by a plurality of feet.

In accordance with another aspect of the invention, there is provided a method of implanting a vascular access device according to the first aspect. The method comprises implanting the components of the first aspect, including the membrane structure of the preceding aspects, the connector arrangement, and the tubular structure. The components may be implanted in pre-assembled form in which two or more of the components are attached to each other or integral with each other. The components may be implanted separately.

In accordance with another aspect of the invention, there is provided a method of using a vascular access device according to the first aspect. The method may comprise using the tubular structure as a fluid channel for the introduction of fluid into the vessel. The method may comprise using the tubular structure to remove fluid from the vessel. The method may comprise using the tubular structure as an access channel for a medical device to provide access into the blood vessel. The method may comprise using the blood vessel as access channel to an organ. The medical device may be a cannula, catheter, diagnostic tool, sensor, or treatment device. For instance, the medical device may comprise a camera, clamp, tissue-sampling device, single-lumen, dual-lumen or multi-lumen flow channels, or similar devices.

In accordance with another aspect of the invention, there is provided a method of removing part of all of a vascular access device in accordance with the first aspect. The method may comprise removing the tubular structure and optionally the connector arrangement from the blood vessel. The method may comprise a step of closing the hole. The method may comprise a step of administering a healing-promoting agent such as a coagulating agent, e.g. thrombin or a precursor of it. The method may comprise the step of using sutures to close the hole. The method may comprise the step of using a plug and/or adhesive to close the hole. The method may comprise leaving the membrane structure in situ, an optionally may comprise using the membrane structure as support for sutures, a plug and/or adhesive.

It is an aspect of the vascular access device that at least some of its components may be used during a medical intervention and also as a vascular closure device at the conclusion of the intervention.

### DESCRIPTION OF THE FIGURES

Exemplary embodiments of the invention will now be described with reference to the Figures, in which:

FIG. 1 is a schematic illustration of an access tube with a connector element;

FIG. 2 is a schematic illustration of an access tube with an umbrella-type membrane in collapsed condition;

FIG. 3 is a schematic top view of an umbrella-type membrane in expanded condition;

FIG. 4 is a schematic illustration of the FIG. 2 access tube with its umbrella-type membrane in expanded condition;

FIG. 5 is a schematic illustration of an access tube in a step of an exemplary use scenario;

FIG. 6 is a schematic illustration of the FIG. 5 access tube in a step of an exemplary use scenario;

FIG. 7 shows a schematic section of an access tube in use;

FIG. 8 illustrates steps during a removal procedure of the access tube; and

FIG. 9 shows steps in a sequence of a method of using a vascular access device.

### DESCRIPTION

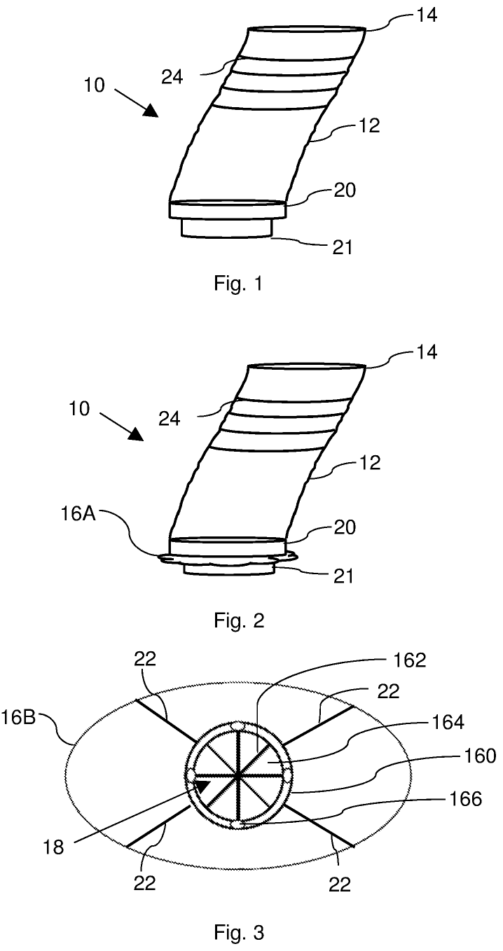

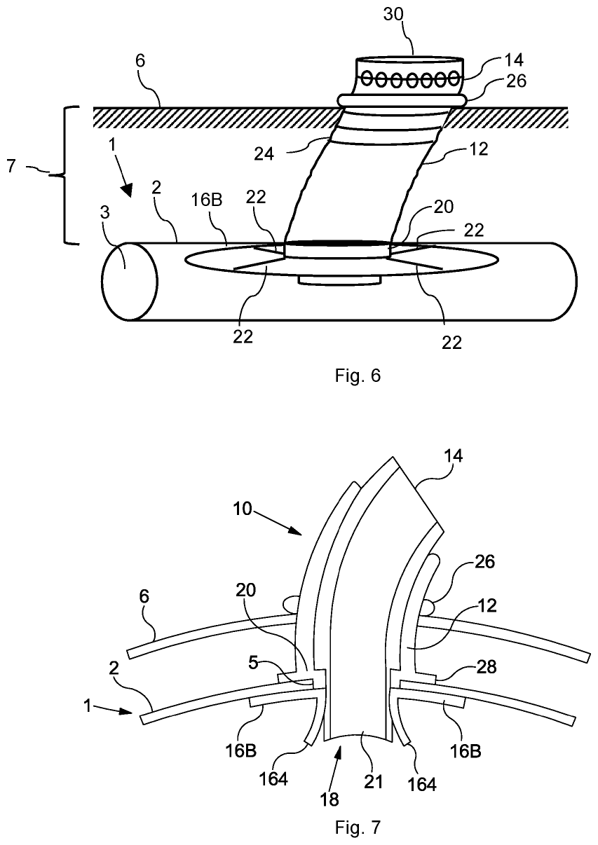

FIG. 1 shows a vascular access tube 10 constituting a tubular structure. The vascular access tube comprises a tube 12 with an external opening 14 and a connector 20 with a connector end 21. Along the outer surface of the tube 12 a plurality of grooves 24 are provided that extend circumferentially around the tube 12 and are spaced apart along the axial extent of the tube 12. The grooves 24 constitute engagement features that will be described in more detail in relation to FIG. 6 below.

FIG. 2 shows a variant of the vascular access tube 10, comprising a tube 12 with an external opening 14 and a connector 20 with a connector end 21, and additionally comprising a collapsed umbrella membrane 16A. The umbrella membrane 16A constitutes a membrane structure. It may be integral with the connector 20 and/or releasably attached, or irreversibly attached to the connector 20. The umbrella membrane 16A comprises a central opening (not depicted in FIG. 2) through which the connector end 21 of the connector 20 protrudes, such that the umbrella membrane 16A is disposed around the connector 20.

FIG. 3 shows a top view of the umbrella membrane 16B in an expanded condition. The umbrella membrane 16B is provided by a patch that has a generally oval circumference and flat profile so as to be able to curve to lie tightly conforming against the inner surface of a blood vessel. Centrally within the umbrella membrane 16B there is provided a closable membrane aperture 18 that is defined by four cuts 162 crossing each other in the manner of circle diameters and separating from one another eight triangular segments providing flaps 164. It can be imagined that each one of the triangular flaps 164 may be bent thereby opening the membrane aperture 18 in the umbrella membrane 16B. The triangular flaps are disposed within an annular coupling structure 160. The coupling structure 160 provides some degree of reinforcement to avoid any of the flaps 164 tearing further than intended by the cuts 162, although it will be understood that depending on the configuration of the flaps a reinforcing effect may not be strong in all embodiments. Radially spaced apart on the coupling structure 160 are several (here: four) alignment points 166 constituting part of a connector arrangement assisting with the alignment of the tubular structure on the membrane aperture 18.

Although depicted centrally the membrane aperture 18 may be located at a different location of the membrane. It will be understood that a different number of flaps may be provided, for instance a single flap e.g. defined by a C-shaped cut, or two or more flaps. The flaps need not have the same size and/or shape.

The arrangement of FIG. 3 illustrates a design that allows providing flaps 164 that are integral with the membrane structure 16B. However, the flaps need not be integral and can be of any shape, e.g. could be larger than the membrane aperture 18 and/or at least partially overlapping each other. The edges of the flaps 164 may be provided with mutually engaging features such as a tongue and groove profile or chevron profile to improve the seal between flap edges when closed. The flaps may comprise a shape memory material such as Nitinol, preferably embedded within the flap structure, to facilitate the return of the flaps into a closed position.

The umbrella membrane 16B comprises a plurality of (here: four) spokes 22. The spokes help to hold the umbrella membrane 16B in an expanded condition and are an example of an anchoring mechanism to provide anchoring to the umbrella membrane against the inside of a vessel by resisting the pulling-out of the membrane. Although four spokes 22 are illustrated in FIG. 3, any number of spokes may be provided. Likewise, a different anchoring structure may be provided, such as a ring or a shape memory structure integrated with the umbrella membrane 16B, or a combination e.g. of a ring with one or more spokes.

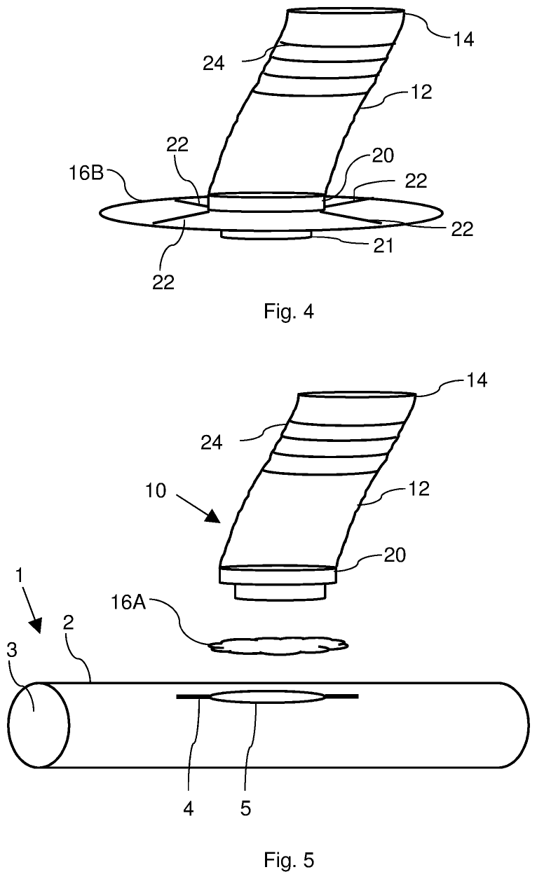

FIG. 4 shows in illustration corresponding to FIG. 2, using the same numerals for similar elements for ease of understanding. In FIG. 4, the umbrella membrane 16B is illustrated in an expanded condition with the connector end 21 inserted through the membrane aperture 18, thereby pushing open the flaps 164 (flaps not depicted in FIG. 4.

FIGS. 1 to 4 are provided to illustrate components of the vascular access device in assembled form to facilitate the understanding of the components. However, the components may be provided separately, for instance as kit of parts, to be assembled in situ. For instance, the umbrella structure may be inserted and expanded before the tube structure is pushed through the membrane aperture.

Turning to FIG. 5, this schematically illustrates parts of the access device prior to implantation onto a blood vessel 1 comprising a vessel wall 2 surrounding a vessel lumen 3. The vessel wall 2 comprises an incision 4 or dilated puncture providing an opening 5 from the exterior into the vessel 1 via the vessel wall 2. The umbrella membrane 16A may be implanted in collapsed condition into the blood vessel 1 via the opening 5 and then expanded and positioned within the blood vessel 1. Although not shown in FIG. 1, an introducer tool may be used to access the blood vessel and/or implant the umbrella membrane 16A. When the umbrella membrane is in place inside the blood vessel 1, the connector end 21 of the access channel 10 is pushed through the flaps 164 of the valve arrangement to open the membrane aperture 18.

Turning to FIG. 6, this shows the umbrella membrane 16B in an expanded condition lying against the inner surface of the vessel wall 2 and surrounding the opening 5 (not shown in FIG. 6). The umbrella membrane 16B is to some extent secured against accidental pulling out from the vessel by way of the anchoring provided by the spokes 22. The tube 12 extends from the blood vessel 1 towards the outside of the skin 6 of a patient, with a length corresponding to a tissue thickness 7. It will be understood that the length of the tube 12 can be adjusted or chosen to suit different level of tissue thickness 7 and may be longer for deeper blood vessels.

A retainer ring 26 is located around the outer circumference of the tube 12 and engaged in one of the grooves 24. The retainer ring 26 helps to limit the extent to which the tube 12 can be pushed into the patient. This reduces the risk of the tube 12 being dislocated and pressing against the blood vessel 1. An external connector 30 is attached to the external opening 14. The external connector 30 may be any suitable connector such as a multi-ridged connector suitable for connecting tubing to form a fluid channel.

FIG. 7 illustrates a section of the vascular access device 10 inserted into a blood vessel 1. For conciseness, the same numerals are used in FIG. 7 as for corresponding elements in the preceding Figures. The umbrella membrane 16B is in an expanded condition lying against the inner surface of the vessel wall 2 such that the flaps 164 of the membrane aperture 18 align with the opening 5 in the vessel wall 2. The connector end 21 of the connector 20 protrudes through the membrane 18 to maintain the flaps 164 in an open position. The connector 20 comprises an annular collar 28 adjacent the connector end 21 which has a wider cross-section than the connector end 21 and so provides a seating surface that prevents insertion into the vessel 1. The connector 20 may be fixed to the membrane aperture 16B by way of a cooperating engagement structure, such as a bayonet connection or friction fit. For instance, the connector end 21 may be dimensioned to tightly fit into the annular coupling structure 160 (not shown in FIG. 7, see FIG. 3) to provide a practically fluid-tight seal. The seating surface 28 and the spokes 22 provide locator structures that help to keep the umbrella membrane 16B and the tube 12 in place and to maintain their position relative to one another. The seating surface 28 may be designed to cooperate with the alignment structures 166 to ensure the correct location of the tubular structure relative to the membrane aperture. The seating surface 28 may comprise or consist of pliable material, for instance in the form of a surface configuration such as a lining. The pliable material may have flexibility sufficient to allow it to conform to the curvature of a vessel wall. The alignment structure may comprise a lip on the seating surface 28. The lip may be integral with the seating surface 28 and comprise or consist of a flexible material able to conform to the shape and wall thickness of the hole in the vessel wall.

As illustrated in FIG. 7, the tube 12 may be provided as a multicomponent wall structure with an inner duct and an outer sleeve. The inner duct may be designed with fluid flow properties in mind. The outer sleeve may be designed with practical aspects of an implant, such as locating features and/or retention features. For instance, the outer sleeve could be overmoulded over an inner duct. However, this need not be the case and in embodiments the tube 12 comprises a single-component wall.

The connector end 21 may be provided with one or more alignment features to align with the corresponding alignment points 166 on the umbrella membrane (see FIG. 3). The coupling between the tube and the umbrella membrane may comprise a magnetic mechanism. The coupling between the tube and the umbrella membrane may comprise an adhesive. A structure similar to the access device 10 illustrated in FIG. 7 may be provided in pre-assembled form, to be implanted with the help of a delivery tool or an introducer device. For instance, a delivery tool may comprise a sleeve in which the umbrella membrane is held in collapsed form and in which the collar providing a seating surface is collapsed. When the vascular access device is inserted through the opening 5 and aligned appropriately across the vessel wall 3, the delivery tool can in that case be removed to let the umbrella membrane expand within the vessel wall and to let the seating surface expand outside the vessel wall.

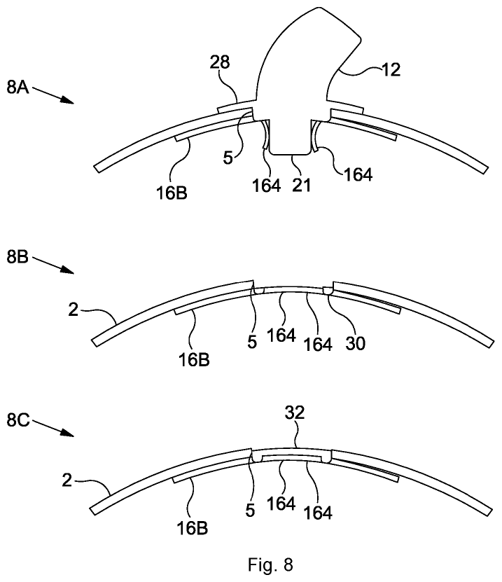

FIG. 8 shows a sequence of three steps during removal of the vascular access device 10 e.g. after conclusion of an intervention. In the top illustration 8A, the tube 12 is located in the opening 5 and the connector end 21 maintains the flaps 164 of the membrane aperture in an open condition. In the middle illustration 8B, the tube 12 has been removed and so the flaps 164 are urged into a closed condition in which they keep the opening 5 in the vessel closed and practically fluid tight. The flaps 14 may comprise a shape memory structure to help ensuring that the flaps are sufficiently flexible to properly close even after prolonged periods of time during which they may have been exposed to the body's immune system. As depicted in illustration 8B, the removal of the tube 12 results in a sealing of the vessel in a manner that is practically fluid tight such that the vessel can continue to supply blood, without a requirement to suture or clamp the vessel at the stage of removing the tube. In embodiments in which the valve arrangement is provided separately from the umbrella membrane, it may be necessary to close the membrane aperture with a separate sealing element, such as a suitable foam or cap. The valve arrangement illustrated in FIGS. 3 and 8B is integral with the membrane structure and thereby avoids a need for introducing a separate vascular closure device after removal of the tubular structure.

At the stage shown in the middle illustration 8B, a new tube 12 or similar device could be reinserted into the membrane aperture. This can be achieved without requiring a new puncture in the vessel wall.

The last illustration 8C of FIG. 8 shows the conclusion of an intervention, where the flaps may be provided with an additional seal, for instance a less reversible seal mechanism, e.g. using sutures 30 or adhesive material 32. The umbrella membrane 16B may be left in place after conclusion of a procedure. For instance, the umbrella membrane may consist of a biocompatible material to be endotheliased (overgrown with vessel-lining tissue).

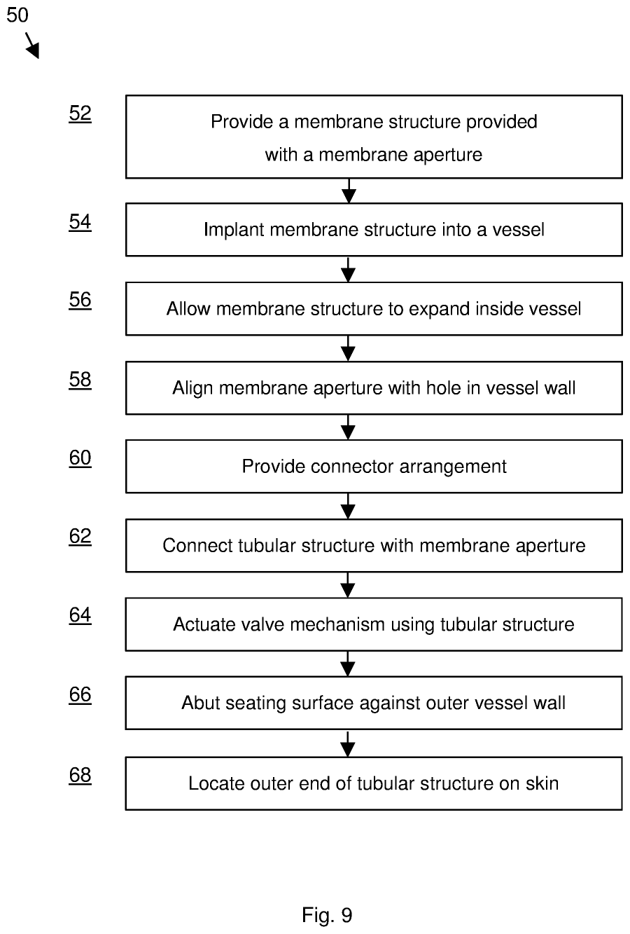

FIG. 9 shows steps of using the vascular access device in a method 50 of accessing a vessel such as the femoral artery or the axillary artery. The method 50 comprises a step 52 of providing a membrane structure with a membrane aperture. The membrane aperture may comprise a valve arrangement to close the membrane aperture. The method may comprise a step 54 of inserting into the vessel, via a hole such as an incision or puncture made in a vessel wall, the membrane structure in an at least partially collapsed condition. In another step 56, the membrane structure is allowed to expand so as to lie against an inner wall of the vessel, surrounding the hole. In another step 58, a membrane aperture is aligned with the hole. Step 58 may precede or may be executed concurrently with step 56. In another step 60, a connector arrangement is provided. The connector arrangement or part of it may be pre-manufactured and/or be integral with the membrane structure. Preferably, the connector arrangement surrounds at least part of the membrane aperture. In another step 62, a tubular structure is provided and attached to the membrane structure via the connector arrangement. The tubular structure may comprise the connector arrangement or a part thereof. In another step 64, an end of the tubular structure is pushed through the membrane aperture to open a valve mechanism. For instance, the end of the tubular structure may push open a flap valve arrangement. Thereby, the valve mechanism is actuated by way of carrying out the attachment of the tubular structure. In another step 66, a seating surface of the tubular structure is abutted against a part of the connector arrangement or the vessel wall to prevent further insertion of the tubular structure into the vessel. In another step 68, an outer end of the tubular structure is located on a patient's skin. It will be understood that some of the steps may be optional, may be carried out in a different order and/concurrently, and that some steps may be carried out implicitly by way of carrying out other steps. For instance, the connector end of the tubular structure may be designed such that the flaps of the valve arrangement are opened and just before the tubular structure connects in a fluid tight manner with a coupling feature of the membrane aperture.

The tubular structure of the access device can be used to introduce fluids directly into the blood vessel. For instance, the access device can be used to introduce oxygenated blood towards a target organ. Likewise, the access device can be used as an access channel for clinical tools.

Removal of the vascular access device may include a step of detaching the tubular structure from the membrane structure. The removal of the tubular structure may allow the valve arrangement to close thereby to close the membrane aperture. In another step, the valve arrangement may be sealed further using a cap, foam or sutures. The membrane structure may remain in place.

The method may be used in a training environment, for instance using phantoms or training material, without providing an actual treatment of a human or animal organism.

The vascular access device combines the ability to provide an access channel into a blood vessel with the ability to seal the blood vessel using a component of the access device. Hitherto, access into a vessel required at least two separate tools, namely a cannula or catheter to be inserted via a puncture in a vessel wall, and a vascular closure device to seal the puncture after a procedure. The present access device integrates a closure functionality with the vascular access channel. This reduces the need for introducing and aligning a further tool upon conclusion of a clinical procedure.

1. A vascular access device for implantation onto a vessel wall, the access device comprising:
a tubular structure to provide an access passage into the vessel via a hole in the vessel wall;
a connector arrangement for attaching the tubular structure at said hole in the vessel wall;
a membrane structure sufficiently flexible to be collapsed for insertion through said hole and to be expanded to lie at least partially against an inner vessel surface thereby to surround the hole, wherein the membrane structure comprises a membrane aperture to provide a passage from inside the vessel into the tubular structure; and
a closing mechanism capable of closing the membrane aperture while the membrane structure is expanded against said inner vessel surface.

2. The vascular access device according to claim 1, wherein the closing mechanism comprises a valve arrangement, wherein optionally the valve arrangement comprises one or more flaps.

3. The vascular access device according to claim 1, wherein at least part of the closing mechanism is provided by a portion of the membrane structure.

4. (canceled)

5. The vascular access device according to claim 1, wherein the device further comprises anchoring structures that are also insertable into the vessel through the hole and are expandable and/or articulated to provide structural support to the membrane.

6. The vascular access device according to claim 5, wherein the anchoring structures are feet or a ring structure.

7. The vascular access device according to claim 1, wherein at least part of the connector arrangement is integral with the membrane structure.

8. The vascular access device according to claim 1, wherein at least part of the connector arrangement is integral with the tubular structure.

9. The vascular access device according to claim 1, wherein the connector arrangement comprises a component detachably connectable to the membrane structure.

10. The vascular access device according to claim 1, wherein the connector arrangement provides an annular port to receive an end of the tubular structure.

11. The vascular access device according to claim 1, wherein the tubular structure comprises an end portion whose outer circumference is smaller than the membrane aperture so as to be insertable through the membrane aperture.

12. The vascular access device according to claim 1, wherein the tubular structure comprises an end portion whose outer circumference corresponds sufficiently closely in shape to the membrane aperture so as to be able to maintain the closing mechanism in a flow-permitting configuration.

13. The vascular access device according to claim 1, wherein the tubular structure comprises around at least part of its outer circumference a seating surface of wider circumference and set apart from an end portion of the tubular structure.

14. The vascular access device according to claim 13, wherein at least part of the connector arrangement is provided by the seating surface.

15. The vascular access device according to claim 11, wherein the tubular structure comprises an end portion whose inner circumference has a lumen diameter sufficient to permit flow rates of at least 1 litre per minute at typical vascular driving pressures.

16. The vascular access device according to claim 11, wherein the end portion comprises along its luminal surface regularly repeating inner wall structures.

17. The vascular access device according to claim 1, wherein the tubular structure comprises on its outer surface a plurality of engagement structures spaced apart along the elongate extension of the tubular structure.

18. (canceled)

19. (canceled)

20. The vascular access device according to claim 1. wherein the closing mechanism comprises a shape memory material.

21. The vascular access device according to claim 2, wherein the valve arrangement comprises one or more flaps, and wherein one or more of the flaps comprise mutually engaging profiles at flap-to-flap edges.

22. (canceled)

23. A method of implanting a vascular access device according to claim 1, the method comprising implanting at a hole in a vessel wall a membrane structure, a connector arrangement, and a tubular structure, wherein the membrane structure is provided with a membrane aperture and inserted through a hole in a vessel wall and expanded to lie against an inner surface of the vessel surrounding the hole, and wherein the method comprises aligning the membrane aperture with the hole, and wherein the method further comprises using a closing mechanism to close the membrane aperture while the membrane structure is expanded against the inner vessel surface.

24. The vascular access device according to claim 9, wherein the component is integral with the tubular structure.
